(19) United States
(12) Patent Application Publication
(10) Pub. No.: US 2008/0199843 A1
(43) Pub. Date: Aug. 21, 2008

(54) Bioartificial Heart Tissue Graft And Method For The Production Therefor

(76) Inventors: Axel Haverich, Hannover (DE); Artur Lichtenberg, Heidelberg (DE); Igor Tudorache, Hannover (DE); Serghei Cebotari, Hannover (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/914,418

(22) PCT Filed: May 17, 2006
(86) PCT No.: PCT/DE06/00850

§ 371 Date: Apr. 17, 2008

(30) Foreign Application Priority Data
May 18, 2005 (DE) 10 2005 023 599.9

(51) Int. Cl. A61L 27/36

(52) U.S. Cl. 435/1.1

(57) ABSTRACT

The invention relates to a method for producing a bioartificial heart tissue graft, which leads to excellent biomechanical properties on the product and guarantees a high cell freedom with optimal preservation of the matrix. During the method, biological cells of a heart tissue preparation, particularly a heart valve or a heart vessel adhering in and/or to an extracellular matrix, are removed in-vitro. The method comprises that following steps carried out in this sequence: a) providing the heart tissue preparation of natural origin; b) removing cells, which are located in the tissue, from the extracellular matrix with the aid of an acellularization solution consisting of an aqueous solution of at least one strong anionic detergent and at least containing sodium deoxycholate; c) osmotically treating the tissue with distilled or deionized water, and; d) treating the tissue with a physiological saline solution with continuous flow or exchanging of the rinsing solution three times.

The invention relates to a bioartificial heart tissue graft and a method for the production thereof.

A bioartificial graft means here a body tissue which is intended for grafting to replace a natural organ, organ part or tissue in humans and is from an allogeneic or xenogeneic source, and which has been made suitable, by removing in particular the cells present thereon, but also other components such as proteins, blood constituents and other immunogenic components, for implantation into another body, and thus represents an originally biological, but manipulated and hence bioartificial product. The bioartificial product may be exclusively deprived of its individual specificity, namely primarily acellularized, or it may have been individually adapted for the intended recipient, e.g. by in vitro colonization with cells which are suitable for the recipient or are the recipient's own, or else by applying a particular coating or treatment intended to facilitate the incorporation in the recipient.

In the present case, the bioartificial product is a bioartificial heart tissue graft.

Numerous methods for acellularization (also called decellularization) of biological tissues are known in the state of the art. These methods generally serve the purpose of making biological grafts more suitable for the recipient. When organs and tissues derived from organ donors (allogeneic grafts, homografts) or of animal origin (xenografts) are grafted, immune rejection reactions occur and may in the worst case lead to loss of the organ or tissue. The graft recipient must therefore take immunosuppressants, which prevent such a rejection reaction, life-long.

In order to preclude rejection of grafts in principle, attempts have long been made to prepare the allogeneic or else xenogeneic material provided therefor for the recipient by firstly removing all immunogenic components, including the cells which are present in the tissues and are foreign to the recipient. Since this includes all living cells, it should simultaneously be able to eliminate effectively contamination by bacteria and viruses, often making the use of grafts of animal origin possible for the first time. Remaining after removal of these cells and other components is ordinarily only the extracellular matrix of the tissue or organ, which consists of the main components, collagen, fibrin and/or elastin and may comprise further structure-providing substances such as hyaluronins and proteoglycans. The extracellular matrix is also referred to as interstitial tissue or interstitial connective tissue.

In early attempts, the acellularized tissues were preserved in the interim before grafting, for example cryopreserved or fixed with glutaraldehyde. However, this led to calcification and other serious disadvantages for the natural incorporation and remodeling of the tissue in the recipient.

Shortly thereafter there was therefore a shift to preparing the acellularized constructs further for the recipient by intending to replace the removed cells by others, preferably by the recipient's own cells of the recipient which had been grown in vitro and were of a type suitable for the respective bioartificial graft. The greatest problem in this connection is to recolonize the graft in a manner resembling nature because complex local structures and a large number of cell types may be involved in nature. However, it was observed that precolonized grafts are remodeled in the body of the graft recipient, so that a mono- or multi-layer initial colonization of certain cells may be sufficient, as described for example in U.S. Pat. No. 6,652,583 (Hopkins), U.S. Pat. No. 5,843,182 (Goldstein) and U.S. Pat. No. 5,899,936 (Goldstein). Nevertheless, a recolonized graft may also lead to problems if, for example, excessive overgrowth of the graft with endogenous cells takes place, so that the geometrical and mechanical properties are influenced too greatly (hyperplasia).

The basis for constructing bioartificial tissue grafts still remains effective acellularization of natural biological material. The acellularization may provide a mechanical and/or chemical removal of the cells. It is not in general possible in a mechanical treatment to spare the extracellular matrix, so that mechanical treatment steps should be confined to removing externally adherent cells or membranes. Chemical acellularization methods therefore predominate by far. Chemical agents used for detaching, or digesting or lysing the cells, are inter alia alkaline solutions, enzymes, glycerol, nonionic and ionic detergents, either singly or in a wide variety of combinations.

It is hoped to obtain very particular effects through the use of particular agents in a particular sequence.

Thus, for example, U.S. Pat. No. 6,448,076 (Dennis et al) prescribes for the acellularization specifically of nerves that the nerve specimen initially be placed in glycerol in order to disrupt the cell membranes, and then intracellular proteins be denatured and removed by placing in at least one detergent solution.

U.S. Pat. No. 6,376,244 discloses the generation of a decellularized kidney matrix by initially placing the kidney specimen in distilled water in order to destroy the cell membranes, and then extracting cellular material with alkaline detergent solution.

To produce a soft tissue graft, U.S. Pat. No. 6,734,018 provides a treatment of the tissue with an extracting solution, a treating solution and a washing solution, where the extracting solution is an alkaline solution with a nonionic detergent and at least one endonuclease, and the treating solution comprises an anionic detergent. The treatment with anionic detergent therein serves not only for acellularization but at the same time to treat the tissue, namely to influence recolonization. It is stated in this connection that treatment with a strong anionic surfactant may lead to interactions with the matrix proteins and to deposition of the surfactant (or detergent) in the acellularized tissue, so that the anionic detergent is retained in the matrix and may remain in the tissue until after grafting. This is on the one hand undesired because of toxic effects associated therewith, but within limits it is also desired in order thus to modulate the recolonization rate, which is retarded by the presence of the detergent.

U.S. Pat. No. 6,734,018 has also dealt in detail with the significance and importance of the sequence of steps. Thus, it is stated that the treatment with SDS before treatment with saline solution leads to different results than does treatment with SDS after treatment with saline solution.

The invention is based on the object of creating a gentle and efficient method for producing a stable, functionally intact, acellularized heart tissue matrix which can be retained as far as possible after grafting. It is moreover the intention that grafting be possible with and without recolonization with the patient's own cells.

To achieve this object, the invention provides in a method for producing a bioartificial heart tissue graft in which biological cells adhering in and/or on an extracellular matrix are removed from a heart tissue specimen, in particular a heart valve or a cardiac vessel, in vitro, for the method to include the following steps in the stated sequence:
a) provision of the heart tissue specimen of natural origin;
b) removal of cells present in the tissue from the extracellular matrix with the aid of an acellularizing solution composed of an aqueous solution of at least one strong anionic detergent which comprises at least sodium deoxycholate;
c) osmotic treatment of the tissue with distilled or deionized water;
d) treatment of the tissue with physiological saline solution.

It has been found that complying with precisely these steps, whose significance will be explained hereinafter, provides particularly good results especially in the acellularization of heart tissues. The method results in acellularized bioartificial grafts which have good mechanical and physiological properties. On the basis of experiments carried out to date, the grafts show promise of being well accepted in the body, even without previous in vitro colonization, and lead to the expectation, because of their biomechanical properties, of long retainability. It is particularly important for use specifically in heart valves that the basement membrane remains intact in the method. In general, the morphology corresponds to a large extent to the natural morphology of the underlying tissue specimen, and a high degree of freedom from cells can be achieved.

The first procedural step in an acellularization method always includes the provision of a natural tissue which may be of allogeneic or xenogeneic origin. If necessary, the natural tissue piece is prepared in the manner most suitable for grafting in the potential recipient. However, there are also specimens which are always suitable for a group of graft recipients, e.g. heart valves in a particular range of ring sizes. The vessel attachment around the valve ring may be configured or cut in different lengths. Step a) of the method therefore includes the selection and cutting of the natural specimen, where appropriate also after intermediate storage, and the introduction into an apparatus suitable for the method or into a suitable vessel, e.g. a dish or bottle.

The heart tissue specimen is preferably a pulmonary valve (valva truncti pulmonalis), an aortic valve (valva aortae), a tricuspid valve (valva atrioventricularis dextra), a mitral valve (valva atrioventricularis sinistra), a valveless vessel piece with or without branch or a pericardial tissue piece for a heart tissue patch.

The acellularizing solution used in the next step of the method (step b) comprises according to the invention strong anionic detergents and of these in each case sodium deoxycholate, because the use of this detergent has proved to be particularly effective for the acellularization in this stage of the method and in association with the other steps.

Besides sodium deoxycholate, at least one further anionic detergent from the group consisting of salts of higher aliphatic alcohols, preferably sulfates and phosphates thereof, sulfonated alkanes and sulfonated alkylarenes, each having 7 to 22 carbon atoms preferably in an unbranched chain, ought to be present because it has surprisingly been found that these mixtures can be washed out of the tissue more easily than said agents alone.

It is currently particularly preferred for the acellularizing solution to comprise besides sodium deoxycholate sodium dodecyl sulfate (SDS), preferably both components in a concentration between 0.05 and 3% by weight, together not more than 5% by weight and in a particularly preferred embodiment in concentrations between 0.3 and 1% by weight, particularly preferably each 0.5% by weight.

The treatment of the natural tissue in such a preferred solution of, for example, 0.5% by weight SDS and 0.5% by weight sodium deoxycholate in water is in principle as known per se for other acellularizing methods. It is preferred and possible for the natural tissue to be treated by shaking or swirling in a container with or without clamping device for the tissue, at room temperature or slightly reduced room temperature, for instance at 15 to 30° C., for about 24 hours, but also longer or shorter depending on the tissue. It is intended that the tissue in this case is completely covered by the acellularizing solution. The acellularizing time can be adapted by the skilled worker with the aid of preliminary tests to the respective tissue which is to be treated, and it is also possible for the acellularizing solution to be changed more than once if this appears advantageous for the respective acellularizing task.

It is true for this as for the other treatment steps that substantially all procedures customary in the prior art can be used, i.e. placing in the respective solutions in dishes or containers, treating in special bioreactors, in part also with pulsatile pressure, rinsing with a solution which is circulated during the treatment period, etc. The solutions can be exchanged one or more times, depending on requirements. Sterile conditions are, of course, employed.

In the next method step (step c) there is osmotic treatment of the tissue with distilled or deionized water for in general at least 20 hours. This step has surprisingly emerged as advantageous in connection with the overall method, although swelling of the matrix, as is inevitable in distilled or deionized water, is not otherwise regarded as advantageous. Treatment with distilled water is known in principle within acellularizing methods, but usually for lysis of the cells, i.e. of the cell membranes, which in this case have for the most part already been removed in the preceding step. Thus, in this step of the invention there is treatment of the extracellular matrix, which is thereby evidently put—in association with the other steps—into a positive state.

In the last step (step d) of the method, the tissue is treated with physiological saline solution, preferably with continuous flow-through or with the rinsing solution being changed at least 3 times. A physiological saline solution means here a substantially isotonic solution which may be in particular a buffered saline solution. PBS or physiological sodium chloride solution is preferably used.

The rinsing preferably with PBS generally takes place by putting the specimen into this solution and shaking or swirling at 15 to 30° C. or room temperature for, preferably, 72 to 96 hours. During this, the solution should be changed at least 3 times, but preferably about 6 to 8 times, for example 7 times. Alternatively, continuous rinsing with PBS flowing through is also possible.

The rinsing should be continued until the remaining concentration of detergent measured in the rinsing solution is zero or no longer cytotoxic.

It is also possible in such methods to add conventional additions such as antibiotics and/or antimycotics to the saline solution in step d).

The bioartificial heart tissue graft can be employed following the method. Until used, it can be stored under cool conditions for some time, during which it should be present in isotonic solution. The graft can also be cryopreserved if direct use is impossible.

The method is further explained by means of experimental examples, with an overview of the biomechanical properties being given in table 1. The biomechanical properties differ distinctly from those which can be achieved with methods which differ in the sequence of steps or the compositions of the solutions.

The bioartificial heart tissue graft can be implanted in the form obtained after the method of the invention. It is assumed according to the current state of knowledge that the acellularization solely or at least substantially with anionic detergent is capable of gently decellularizing specifically a heart tissue matrix, where the matrix proteins are in a charged state such as appears to be optimal for recolonization in the body of the recipient or in vitro, without cytotoxic concentrations of acellularizing surfactant still being present in the matrix. The biomechanical properties are also suitable for direct implantation of the still uncolonized heart tissue graft to be possible.

However, the acellularized heart tissue specimen can also be recolonized with viable biological cells in vitro before grafting, preferably with endothelial cells. The cells used for recolonization in vitro are preferably autologous cells of the potential graft recipient, which have been taken from him in preparation for the grafting and have been grown in vitro. The methods necessary for this are known in the state of the art.

### EXAMPLE

A pulmonary valve (valva truncti pulmonalis) with vessel conduit was removed from a porcine heart and rinsed with PBS solution to remove residues of blood. Incubation in a 0.5% sodium cholate/0.5% SDS solution took place with shaking at 20° C. for 24 hours for the acellularization. The tissue was then incubated with distilled water, shaking at 20° C. for 24 hours. The tissue was finally rinsed in PBS solution, shaking at 20° C. for 96 hours. In this step, the PBS solution was changed every 12 hours.

### Biomechanical Tests:

Samples of porcine pulmonary valve walls (5 samples per investigation group) were cut to a length and width of 15 mm by 10 mm and mounted in clamps specifically constructed for this purpose in such a way that the unloaded reference length of the sample piece, suspended under its own weight, was 10 mm. The cross-sectional area along the reference length defined in this way was determined using a contact-free laser micrometer (LDM-303H-SP, Takikawa Engineering Co., Tokyo, Japan). The samples were cut for testing in the longitudinal and transverse direction in relation to the vessel direction (pulmonary artery) and kept moist with PBS solution during the tests.

The sample pieces were then preloaded with 0.01 newton and gradually lengthened as far as macroscopic failure. This step took place in a material-testing apparatus (model 1445, Zwick GmbH, Ulm, Germany) at a rate of 0.1 mm extension per second. Force-elongation and stress-strain plots were recorded for each tested sample piece, and the limiting stress, structural rigidity, limiting strain of the sample piece (elongation at break) and the modulus of elasticity (Young's modulus) were determined. The structural rigidity and the modulus of elasticity were determined in the linear region of the force-elongation and stress-strain plots. The limiting stress and the breaking stress were taken at the point at which the first significant fall in respectively the tensile stress and the force was evident (table 1).

### Definition of the Measured Parameters Indicated in Table 1

### Breaking Stress F_{R}[N]:

The breaking stress is defined as the force measured at the instant of break. The breaking stress was taken at the point at which the first significant fall in the force was evident.

### Ultimate Tensile Strength δ_{R}[N/mm²]:

The ultimate tensile strength is the quotient of the force F_{R} measured at the instant of break and the initial cross section A₀[N/mm²] [corresponds to MPa]

δ_{R}=F_{R}/A₀[N/mm²] [corresponds to MPa]

### Modulus of Elasticity E [N/mm²]:

The modulus of elasticity is a measure of the strength of a material. It indicates how much a material extends under a particular load.

The modulus of elasticity is defined as the slope in the graph of the stress-strain plot within the elasticity region. Where the stress-strain plot is linear (proportionality region), the following applies:

E=δ/ε[N/mm2] [corresponds to MPa]

In this case, δ designates the (tensile) stress and ε the extension.

TABLE 1
Results of the biomechanical tests

| | Comparative group, | | Trypsion | | NaD group, | |
|---|---|---|---|---|---|---|
| | n = 5 | | group, n = 5 | | n = 5 | |
| | long | trans | long | trans | long | trans |
| Area (mm²) | 29.2 ± 5.7 | 29.6 ± 6.3 | 27.6 ± 5.4 | 28.1 ± 5.9 | 31.4 ± 5.4 | 26.6 ± 5.8 |
| Limiting force | 9.5 ± 4.3 | 13.9 ± 1.5 | 5.0 ± 2.37* | 11.8 ± 3.2 | 9.8 ± 1.3 | 16.2 ± 5.6 |
| (N) | | | | | | |
| Rigidity (N/mm) | 3.0 ± 2.6 | 4.3 ± 1.6 | 2.3 ± 1.5 | 4.2 ± 1.5 | 3.0 ± 0.79 | 7.0 ± 3.0 |
| Elongation at | 0.8 ± 0.48 | 1.6 ± 0.63 | 0.4 ± 0.2* | 0.5 ± 0.2† | 0.5 ± 0.1 | 1.0 ± 0.3 |
| break (mm/mm) | | | | | | |
| Limiting stress | 0.32 ± 0.15 | 0.50 ± 0.18 | 0.18 ± 0.06* | 0.43 ± 0.16 | 0.32 ± 0.05 | 0.61 ± 0.2 |
| (MPa) | | | | | | |
| Young's modulus | 1.04 ± 0.94 | 0.86 ± 0.57 | 0.69 ± 0.5 | 1.69 ± 1.04 | 0.99 ± 0.25 | 1.75 ± 1.35 |
| (MPa) | | | | | | |

| | NaD + SDS | | SDS group, | |
|---|---|---|---|---|
| | group, n = 5 | | n = 5 | |
| | long | trans | long | trans |
| Area (mm²) | 31.8 ± 6.6 | 29.9 ± 1.6 | 26.3 ± 4.0 | 26.8 ± 4.88 |
| Limiting force | 6.6 ± 2.1 | 11.4 ± 3.9 | 7.9 ± 5.6 | 15.5 ± 5.8 |
| (N) | | | | |
| Rigidity (N/mm) | 2.7 ± 0.7 | 3.9 ± 1.25 | 2.6 ± 1.4 | 5.3 ± 1.5 |
| Elongation at | 0.6 ± 0.2 | 1.2 ± 0.08 | 0.7 ± 0.5 | 0.7 ± 0.4* |
| break (mm/mm) | | | | |
| Limiting stress | 0.22 ± 0.11 | 0.39 ± 0.14 | 0.29 ± 0.11 | 0.57 ± 0.13 |
| (MPa) | | | | |
| Young's modulus | 0.90 ± 0.38 | 1.18 ± 0.55 | 0.97 ± 0.52 | 2.06 ± 0.84* |
| (MPa) | | | | |

Data indicated as mean ± standard deviation
*< 0.05,
† p < 0.01,
§p < 0.001 versus comparative group

The test results are elucidated by means of the figures. These show:

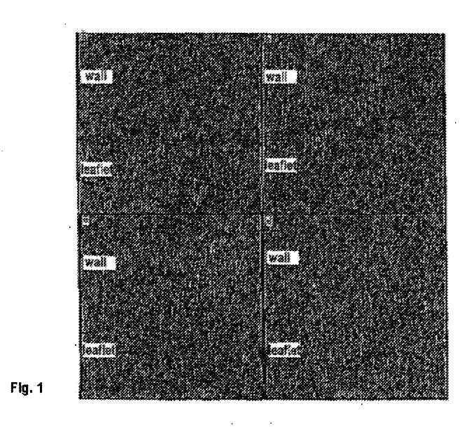

FIG. 1a) valve treated with trypsin solution (0.05% trypsin/0.02% EDTA);

FIG. 1b) valve treated with sodium deoxycholate solution (1% strength);

FIG. 1c) valve treated with sodium deoxycholate and SDS (each 0.5% strength solutions)

FIG. 1d) valve treated with SDS solution (1% strength)

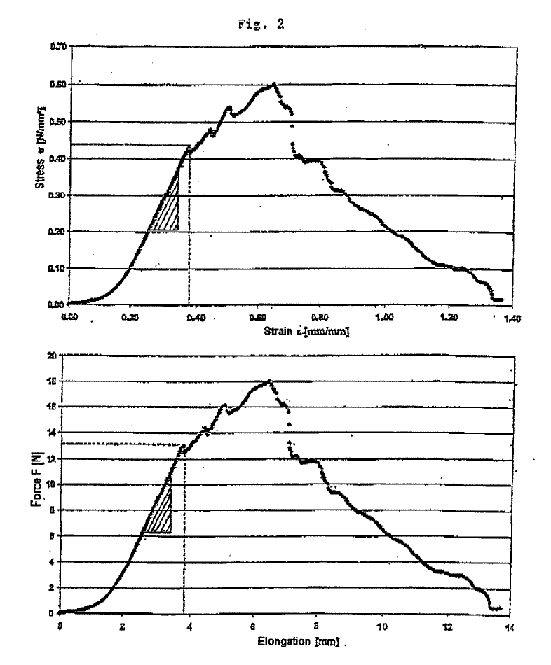

FIG. 2) stress-strain plots (top) and force-elongation plots (below).

FIGS. 1a) to 1d) show scanning electron micrographs of porcine pulmonary valve regions treated with various acellularizing solutions (the method being otherwise the same). In each case an intraluminal surface with transverse section of wall (at the top in each picture) and leaflet (at the bottom in each picture) is shown.

As is evident, the trypsin treatment greatly damages the basement membrane. It is apparent that the method of the invention and sodium deoxycholate in the acellularizing solution give good results.

The biomechanical properties for these experiments are quantified in table 1. Typical stress-strain plots and force-elongation plots are shown in FIG. 2. The shaded slope triangles characterize the regions in which the modulus of elasticity and the structural rigidity are calculated. The point on the curve characterized by dotted lines characterizes the limiting stress, the limiting strain and the breaking stress.

It emerges that the rigidity in the longitudinal and transverse direction is changed by not more than about +−15% compared with native comparative tissues. The Young's modulus is also increased only moderately compared with native comparative tissues. The biomechanical properties of the product of the method can therefore be designated overall as good.

1. A method for producing a bioartificial heart tissue graft in which biological cells adhering in and/or on an extracellular matrix are removed from a heart tissue specimen, in particular a heart valve or a cardiac vessel, in vitro, where the method includes the following steps in the stated sequence:
a) provision of the heart tissue specimen of natural origin;
b) removal of cells present in the tissue from the extracellular matrix with the aid of an acellularizing solution composed of an aqueous solution of at least one strong anionic detergent which comprises at least sodium deoxycholate;
c) osmotic treatment of the tissue with distilled or deionized water;
ci) treatment of the tissue with physiological saline solution.

2. The method as claimed in claim 1, characterized in that the provided heart tissue specimen is a pulmonary valve (valva truncti pulrnonaljs), an aortic valve (valva aortae), a tricuspid valve (valva atrioventrjcularis dextra), a mitral valve (valva atrjoventrjcuiarjs sinistra), a valveless vessel piece with or without branch ora pericardial tissue piece for a heart tissue patch, in each case of allogeneic or xenogeneic origin.

3. The method as claimed in claim 1, characterized in that, besides sodju.rn deoxycholate, the acellularizing solution comprises at least one further anionic detergent from the group consisting of salts of higher aliphatic alcohols, preferably sulfates and phosphates thereof, sulfonateci alkanes and sulfonated alkylarenes, In each case having 7 to 22 carbon atoms in a preferably unbranched chain.

4. The method as claimed in claim 1, characterized in that, besides sodium deoxycholate, the acellularizing solution comprises sodium dodecyl sulfate (SDs), preferably both components in a concentration between 0.05 and 3% by weight, together not more than 5% by weight, further preferably between 0.3 and 1% by weight, particularly preferably each 0.5% by weight.

5. The method as claimed in claim 1, characterized in that the method is carried Out at temperatures between about 15 and 30° C.

6. The method as claimed in claim 1, characterized in that for treatment steps a) to d) the tissue is put into the appropriate treating solution in a container with or without clamping device for the tissue specimen and is Completely covered by this solution, with the container being shaken or swirled preferably during the treatment period or parts of the treatment period.

7. The method as claimed in claim 1, characterized in that the saline solution in step d) is a buffered saline solution, preferably PBS or physiological sodium chloride solution.

8. The method as claimed in claim 1, characterized in that the saline solution in Step d) is renewed at least 3 times, preferably at least 6 times, or in that the PBS solution flows through continuously.

9. The method as claimed in claim 1, characterized in that antibiotics and/or antimycotics are added to the saline solution in step d).
